(19) United States
(12) Patent Application Publication
(10) Pub. No.: US 2010/0049154 A1
(43) Pub. Date: Feb. 25, 2010

(54) Elastically deformable sanitary napkin

(76) Inventor: Judy Burd, Phillipsburg, NJ (US)

Correspondence Address: THOMAS J. GERMINARIO, ESQ., 154 ROUTE 206, CHESTER, NJ, 07930, US

(21) Appl. No.: 12/229,337

(22) Filed: Aug. 22, 2008

(51) Int. Cl. A61F 13/15

(52) U.S. Cl. 604/379; 604/378; 604/385.01

(57) ABSTRACT

A dynamically deformable sanitary napkin for insertion within a person's intergluteal cleft has a plurality of functional layers serving absorbent, protective, and mechanical purposes and is held in position by the restorative force of a spring means.

### BACKGROUND OF THE INVENTION

The present invention relates to medicinal and hygienic devices used to absorb exudates and/or release medications in the intergluteal and/or perianal areas of a patient or individual.

The area of the human body between the buttocks is inherently susceptible to a number of pathological conditions, including rashes, sores and hemorrhoids. Involuntary anal secretions and exudates also create hygienic problems and are a source of embarrassment. Bodily fluids and gases escaping form the anal orifice are apt to soil garments and produce offensive malodors.

The prior art in this field has developed three basic approaches to anal hygienic expedients: (1) diaper-like undergarments, (2) adhesive pads, and (3) anal plugs. Examples of the first category are taught by Edwardsson, U.S. Pat. No. 6,441,268 and Weimer, U.S. Pat. No. 5,593,398. The absorbent article disclosed by Edwardsson is designed to be worn as an undergarment in a pants-like configuration, much like a disposable baby diaper. The diaper does not project into the intergluteal cleft, but rather covers it and extends above and below it to the sacral and perineum areas, respectively. Weimer describes protective underpants made of air-tight nylon, with a sewn-in absorbent pad that extends over the intergluteal cleft and across the perineum of the wearer.

There are a number of undesirable features of these diaper/underwear articles. While they may be suitable for sedentary elderly and infirm patients with chronic anal incontinence, they are ill-suited to active people who will find these garments confining, uncomfortable, and inconvenient. A working person or student, for example, would have to carry around extra diapers or underpants in case the ones he/she was wearing became soiled. “Changing one's own diaper” is hardly something that an active adult or mature youth would want to do on a regular basis.

Another drawback of the diaper/underpants designs is their lack of versatility. While they deal with the problems of anal exudates and flatulence, they cannot be adapted to address other anal/perianal pathologies, such as dermatitis, abscesses or lesions. Since they do not extend into the intergluteal cleft, their absorbent pads come into contact only with the outer portion of the buttocks. Therefore, they cannot be used to dispense medication to the intergluteal, perianal or anal areas.

Illustrating the adhesive pad type of anal hygienic article are Yabrov et al., U.S. Pat. No. 4,880,417, Conant et al., U.S. Pat. No. 6,313,371, and Hansen et al., International Publication No. WO 2007/134600. In the Yabrov invention, the anal pad has an adhesive layer on its outer surface for attaching it to a user's underwear. In the Conant patent, the pad is taped inside briefs or panties using double-sided adhesive tape. The anal patch taught by Hansen has an adhesive surface that is applied directly to the skin of the buttocks. The primary drawback of these adhesive anal pads is the difficulty of keeping the pad securely in position, especially for active individuals. The gluteus muscles are central to human locomotion. They move in relation to each other and in relation to undergarments each time a person walks, stands, sits, squats, crosses their legs, etc. The position of a pad attached to underpants will constantly shift with respect to the anus in the course of the wearer's activities, necessitating frequent, annoying adjustments. Attachment to the skin, as taught by Hansen, is even more problematic, since the anal area is typically moist and often hairy. The lack of reliability in securing the adhesive pads to a specific area of the buttocks also makes them unsuitable for dispensing medication to localized pathologies, such as rashes, sores or hemorrhoids.

The difficulty of keeping an anal napkin securely in place between the buttocks also besets the third category of prior art, the anal plugs. The simplest of these is taught by Flanders, U.S. Pat. No. 2,742,042. The Flanders napkin is an absorbent pad of uniform thickness designed to be inserted edgewise into the intergluteal cleft. While the Flanders specification asserts that the gripping action of the opposing buttocks is adequate to hold the napkin in place when the user is standing, the buttocks will spread apart when the user sits or squats down, thus releasing their grip on the pad. Somewhat more sophisticated are the anal plugs disclosed by Fleming, Pub. No. US 2005/0182376, which have an ovoid cross-section, such that the smaller minor portion engages the anal orifice, while the larger major portion engages the buttocks. Here again, the mechanics of sitting/squatting are likely to dislocate the plug as the buttocks spread apart. Sitting may also cause the Fleming plug to be pushed against the anal orifice, causing discomfort.

Grosse, U.S. Pat. No. 5,665,081, teaches an anal pad having an wedge-shaped insert that extends into the intergluteal cleft and engages the perianal area. As in the Flanders and Fleming pads, however, the insert had no ability to expand as the intergluteal cleft spreads apart in the seated or squatting postures. A similar wedge-shaped insert is disclosed by Matrullo, U.S. Pat. No. 4,182,335. In Matrullo, the insert is held is place by the mechanical adherence of an outer layer of fluffy material to the wearer's buttocks. While such fluffy material may have a marginal capacity to “fluff out” as the buttocks spread apart in sitting/squatting, repeated expansion and compaction will quickly loosen the fibrous adherence and allow the insert to shift about.

The present invention is a sanitary napkin that is inserted into the intergluteal cleft and remains securely in the desired position without the support of undergarments, adhesives or plugs. This is accomplished by producing a sanitary napkin that is elastically deformable, so that it conforms to the dynamic contours of the intergluteal cleft, not merely to the static contours of a single posture, as the prior art does. The present invention achieves this dynamic conformity by using two opposing wings to engage the inner buttocks. The dual wings are dynamically urged against the buttocks by the action of one or more spring means, which may be distinct elements of the napkin or alternately may be integral to the overall structure of the napkin itself. Due to the spring-action design of this sanitary napkin, the wings of the napkin will spread apart as the buttocks spread apart while sitting or squatting, and they will contract together as the buttocks press together while standing or walking.

In addition to presenting solutions to problems of napkin positioning and stability that have eluded the prior art, the present invention is far more versatile that the other inventions. As will subsequently be explained in greater detail, the dynamic dual-wing design allow this sanitary napkin to be precisely positioned in various areas between the buttocks, so that it can address localized pathologies, such as perianal rashes or lesions, in addition to controlling anal exudates. The dual wings can have several different profile configurations, so that the napkin will cover only those intergluteal areas that need to be kept dry and/or medicated. This versatility minimizes the discomfort and inconvenience associated with using the napkin, and thus makes it more likely that the patient will continue using it, thereby enhancing its hygienic and medicinal efficacy.

### SUMMARY OF THE INVENTION

An object of the present invention is to provide a sanitary napkin for use in the anal/perianal area that is elastically deformable and dynamically conformable to the contours of the intergluteal cleft throughout the full range of gluteal displacement in sitting, walking, running, squatting, etc.

Another object of the present invention is to provide a sanitary napkin for use in the anal/perianal area that effectively absorbs and stores anal exudates and other bodily secretions/excretions, prevents the release of the absorbed and stored fluids, and protects the user and his/her garments from exposure to such fluids.

Yet another object of the present invention is to provide a sanitary napkin for use in the anal/perianal area that can be impregnated with ingredients which act as antiperspirants, anti-irritants, anti-inflammatories, topical anesthetics, antibiotics, bactericidal agents, deodorizing agents, or any other pharmacologically active compound selected to correspond to the needs of a given patient or patient group.

Still another object of the present invention is to provide a sanitary napkin for use in the anal/perianal area that can be precisely positioned so as to engage only those gluteal areas in need of absorbency and/or medication, and so as to be able to treat localized conditions of anal/perianal pathologies, such as dermatitis, lesions, abscesses or hemorrhoids.

A further object of the present invention is to provide a sanitary napkin for use in the anal/perianal area that will minimize the discomfort and inconvenience of the user/patient through a variety of profile configurations, which allow the napkin to be minimally intrusive.

Yet a further object of the present invention is to provide a sanitary napkin for use in the anal/perianal area that can be readily and inconspicuously inserted and removed without manual contact with absorbed bodily fluids.

Still a further object of the present invention is to provide a sanitary napkin for use in the anal/perianal area that is simple to use, inexpensive and disposable.

All of these and other beneficial objectives are achieved by an elastically deformable sanitary napkin comprising dual wings joined at a medial axis. Coincident with the medial axis is/are one or more spring means that maintain a dynamic tension tending to restore the dual wings to an angular separation corresponding to the expanded form of the napkin. In the napkin's expanded form, the spread between the two wings is greater than the width of the intergluteal cleft, thereby allowing the napkin to be held in place by the outward force of the two wings against the buttocks.

The sanitary napkin is placed between the buttocks by manually compressing the two wings together until the spread between them is slightly less than the width of the intergluteal cleft. The napkin in its compressed form is then inserted into the intergluteal cleft, and the manual pressure on the two wings is released, thereby allowing the wings to expand slightly and engage the buttocks. The dual wings are dynamically urged against the buttocks by the elastic restorative force of the spring means. As the buttocks move in the acts of standing, sitting, and walking, the spring means causes the spread between the dual wings to continuously adjust to the changing width of the intergluteal cleft.

The spring means can be one or more specific components of the sanitary napkin, or they can be an elastic deformable characteristic of the napkin structure as a whole.

The two wings of the napkin comprise an outer absorbent layer, which will be in contact with the user's skin, and an inner impervious protective layer, to prevent leakage of absorbed fluids that may soil the user's garments. The absorbent layer can consist of two or more sub-layers having varying degrees of porosity, so that the smaller-pored outer layers closer to the skin have a greater wicking effect, while the larger-pored inner layers have more fluid storage capacity. The varying porosity of the sub-layers can also provide varying release rates for medications dispensed by the napkin.

The dual wings of the napkin will have a variety of profiles designed to conform to different anatomical types as well as specific medical indications. The napkin has an outer crease along its medial axis which can be straight or concave, depending on the contours of the wearer's intergluteal cleft and/or the specific perianal area sought to be engaged by the napkin. Similarly, the edges of the two wings can be straight or convex, depending on the specific intergluteal areas to which absorbency and/or medications are to be applied.

An optional retrieval implement, such as a string or cord, can be attached to one of the napkin's wings to allow the user to remove the napkin from between his/her buttocks without unpleasant manual contact with the absorbent areas of the napkin.

### BRIEF DESCRIPTION OF THE DRAWINGS

FIG. 1 is a perspective view of the present invention.

FIG. 2 is a cross-sectional view, showing the present invention in its expanded form, along line 2-2 of FIG. 1.

FIG. 3 is a cross-sectional view, showing the present invention in its compressed form, along line 2-2 of FIG. 1.

FIGS. 4A and 4B are cross-sectional cut-away views, showing two alternative laminate layer configurations corresponding to embodiments (I) and (II) of the present invention, along line 3-3 of FIG. 2.

FIGS. 5A, 5B, 5C and 5D are overhead plan views of four alternative spring configurations corresponding to embodiments (III), (IV), (V) and (VI) of the present invention.

FIGS. 6A, 6B, 6C, and 6D are left side elevation views of four alternative profile configurations corresponding to embodiments (VII), (VIII), (IX), and (X) of the present invention.

### DETAILED DESCRIPTION OF THE PREFERRED EMBODIMENTS

The present invention will now be described more fully hereinafter with reference to the accompanying drawings in which alternative preferred embodiments of the invention are shown. The invention may, however, be embodied in many different forms and should not be construed as limited to the embodiments set forth herein; rather, these embodiments are provided so that this disclosure will be thorough and complete and will fully convey the scope of the invention to those skilled in the art. This Detailed Description of the Preferred Embodiments incorporates by reference the Summary of the Invention set forth above.

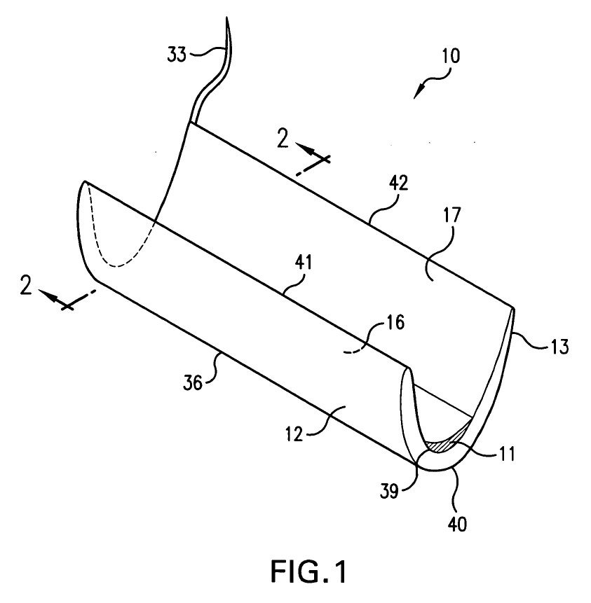

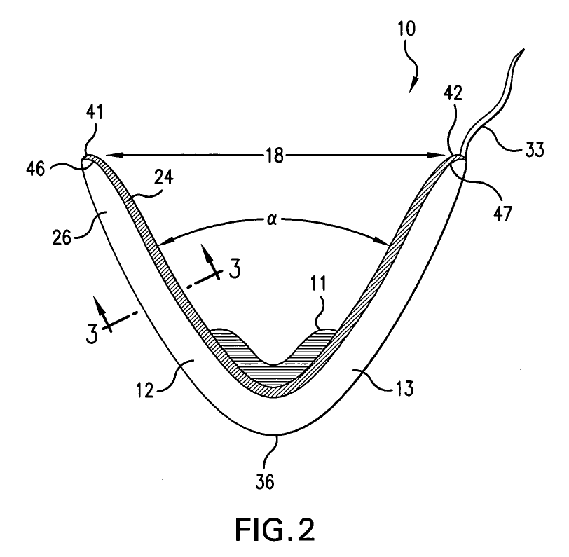

As depicted in FIG. 1, the present invention 10 is a semi-rigid, plastically deformable sanitary napkin that has a cross-section and profile such that it can comfortably be inserted and retained in the intergluteal cleft between the buttocks of a patient. As shown in FIG. 2, the sanitary napkin 10 has a “V” or “U” shaped cross-section that approximates an apex segment of an acute or obtuse angle, an ellipse, a parabola, or a hyperbola. The specific shapes, materials, and pharmaceutical agents used in the present invention 10 can be selected to conform and respond to the anatomy, physiology and pathology of a patient's buttocks and intergluteal area.

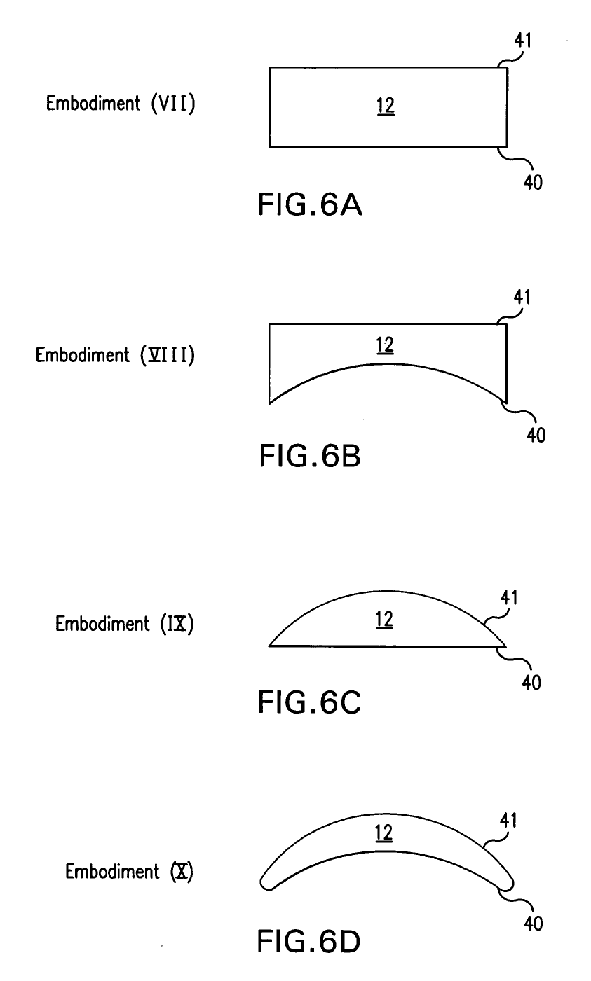

As illustrated in FIGS. 1 and 2, the present invention 10 comprises two opposed wings 12 and 13 that spread apart at an angle a from a medial axis 36. The wing edges 41 and 42 may have cross-sections that are pointed, rounded or blunt, as shown in FIGS. 1 through 3, or they may be flanged (not shown). FIG. 2 depicts the expanded form of the present invention 10, which is to say, the form the napkin assumes when its wings 12 13 are not subjected to a compressing force or restraint. In the napkin's expanded form, the wings 12 13 open to a full width 18 that is greater than the width of the patient's intergluteal cleft. The napkin 10 is bent along the entire length of its medial axis 36, thereby creating an inner crease 39 along its inner surface and an outer crease 40 along its outer surface. Within the inner crease 39 are one or more spring means 11, which are undeformed in the napkin's expanded form. As shown in FIGS. 6A through 6D, the wing edges 41 42 can be straight or convex and the outer crease 40 can be straight or concave, so that the side profile of the napkin 10 can have any one of four alternative shapes, ranging from rectangular to taco-shaped, based on the needs and anatomy and needs of a given patient.

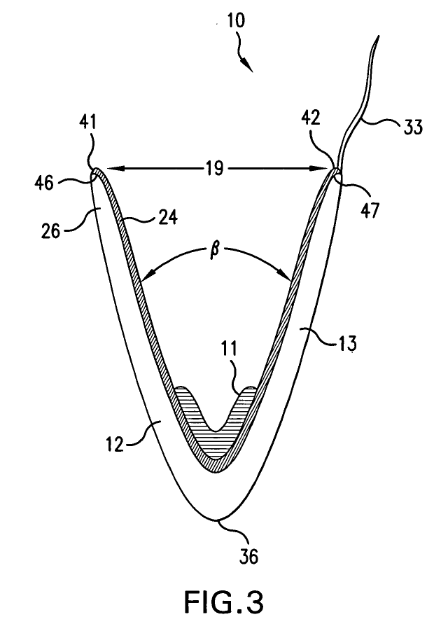

FIG. 3 depicts a cross-section of the present invention 10 in its compressed form, which is to say the form the napkin assumes when its wings 12 13 are subjected to compression upon being inserted into the intergluteal cleft between the patient's buttocks. Comparing the cross-section of FIG. 3 with that of FIG. 2, it can bee seen that the wings 12 13 have been forced closer together in the napkin's compressed form, such that the angle β between the wings is less than the corresponding angle α in the napkin's expanded form. Consequently, in the napkin's compressed form, the wings 12 13 contract to a reduced width 19 that matches the width of the patient's intergluteal cleft. The compression of the wings 12 13 between the patient's buttocks causes the spring means 11 to deform, such that the spring means 11 exerts an outwardly-directed restorative force on both wings 12 13, which restorative force urges the wings 12 13 against the patient's inner buttocks, thereby firmly securing the napkin within the intergluteal cleft.

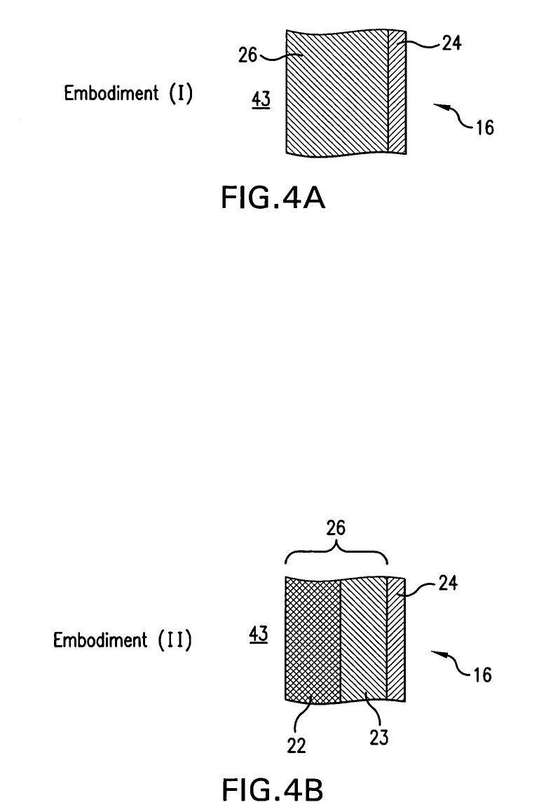

FIGS. 4A and 4B depict two alternative cross-sectional cut-away views of a portion of the napkin 10 in contact with the skin of the patient's inner buttock 43. Viewing FIGS. 4A and 4B in relation to FIGS. 1 and 2, it can be seen that inner surfaces 16 and 17 of the wings 12 13 are covered with a protective layer 24, which is a non-porous, impervious layer that effectively prevents bodily fluids from passing through the napkin 10 to soil the patient's garments or release malodors. The protective layer 24 is bonded to an absorbent layer 26 comprising the outer layer of the napkin 10. In embodiment (I), as shown in FIG. 4A, the absorbent layer 26 is comprised of a homogenous absorbent material, such as a foam, cotton or gauze, which acts as both a wicking and storage layer for bodily fluids. Alternatively, in embodiment (II), the absorbent layer 26 is comprised of two or more foam sub-layers having different cell sizes or porosities. In the version depicted in FIG. 4B, an outer wicking sub-layer 22 is comprised of a foam material having relatively small pores that efficiently wick moisture away from a patient's skin 43, while an inner storage sub-layer 23 has larger pores that effectively store the moisture away from the patient's skin 43.

Any of the sub-layers which comprise absorbent layer 26 may be doped or impregnated with ingredients which act as antiperspirants, anti-irritants, anti-inflammatories, topical anesthetics, antibiotics, bactericidal agents, deodorizing agents, or any other pharmacologically active compound selected to correspond to the needs of a given patient or patient group. In embodiment (II) the wicking sub-layer 22 may be so doped for immediate or rapid release of the doping agent, while storage sub-layer 23 or subsequent inner sub-layers may be so doped as to provide a slower or more controlled sustained release of the doping agent. The pharmacologically active compound can be such that the release of the pharmaceutical agent only occurs in the presence of a pre-determined condition such as a specific temperature, pH, or in the presence of moisture.

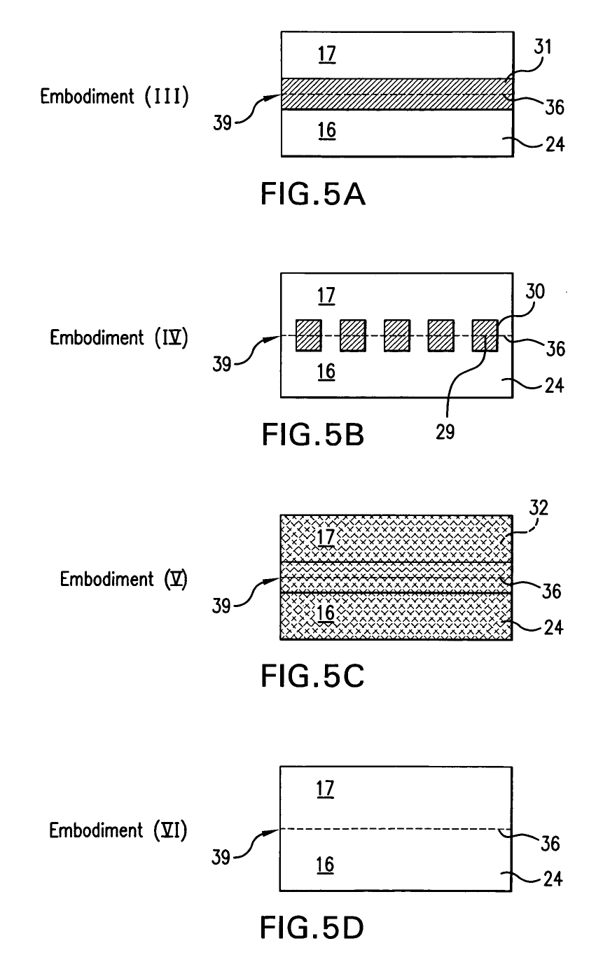

The present invention 10 is semi-rigid and maintains a “V” or “U” shaped cross-section in one of four ways hereafter referred to as embodiments (III), (IV), (V) and (VI), as depicted in FIGS. 5A, 5B and 5C. The shape and deformation properties of the napkin 10 are such that, when it's inserted into the intergluteal cleft between the buttocks, the napkin plastically deforms to the shape of the intergluteal cleft. This deformation is controlled by a deformable, semi-rigid spring means 11, which defines the napkin's geometric and mechanical properties. FIGS. 5A, 5B, 5C and 5D are overhead plan views of three alternative configurations of the spring means 11, which configurations correspond to embodiments (III), (IV), (V) and (VI) of the present invention.

In embodiment (III), as depicted in FIG. 5A, the spring means 11 comprises one deformable, semi-rigid, oblong spring member 31 that is bonded or otherwise attached to the wings 12 13 and extends along the length of the inner crease 39. As shown in FIG. 2, the undeformed cross-section of the oblong spring member 11 conforms to the cross-section of the napkin's inner crease 39 in its expanded form. The oblong spring member 31 is oriented so that its long axis coincides with the medial axis 36 of the napkin 10.

In embodiment (IV), as depicted in FIG. 5B, the spring means 11 is comprised of a plurality of small “V” or “U” shaped spring clips 30 that are uniformly distributed along the inner crease 39 of the napkin 10. Each spring clip is bonded or otherwise attached to the wings 12 13 such that its pivot point 29 aligns with the medial axis 36. As shown in FIG. 2, the undeformed cross-section of the spring clips 11 conforms to the cross-section of the napkin's inner crease 39 in its expanded form.

In embodiment (V), as depicted in FIG. 5C, the spring means 11 comprises a semi-rigid, elastic, deformable layer 32 which is inlaid or laminated into the sanitary napkin between protective layer 24 and absorbent layer 26.

In embodiment (VI), as depicted in FIG. 5D, the spring means 11 does not comprise a separate and distinct structure or structures within the napkin 10, but rather comprises the integral structure of the napkin itself. The napkin's deformable, semi-rigid properties are achieved by laminating the absorbent layer 26 and protective layer 24 when the entire napkin 10 is already in its expanded form. When the absorbent 26 and protective 24 layers are adhered using chemical or mechanical means, the final laminated assembly has the shape of the napkin 10 in its expanded form, and the napkin 10 as whole acts as the spring means 11 by resisting deformation from its expanded form.

The wings 12 13 of the napkin 10 can have a variety of profiles to suit the anatomy and pathology of the patient. FIGS. 6A, 6B, 6C, and 6D are left side elevation views of four alternative profile configurations, which correspond to embodiments (VII), (VIII), (IX), and (X) of the present invention. The alternative profiles are rectangular (VII), rectangle with one concave side (VIII), taco-shaped (IX), and banana-shaped (X). In each embodiment, the outer crease 40 has a contour which allows it to fit more or less snugly against the innermost area of the patient's intergluteal cleft, while the contour of the wing edge 41 allows the wing 12 to engage more or less of the patient's buttock.

It will be appreciated that the ten embodiments of the present invention 10 described hereinabove can be combined with one another in various permutations. Each one of the four profile embodiments (VII)-(X) can be combined with each the four spring embodiments (III)-(VI) to yield sixteen combined profile/spring embodiments. Each of the sixteen combined profile/spring embodiments can, in turn, have either of the two alternative laminate layer configurations in accordance with embodiments (I) and (II). Therefore, there are a total of 32 combined embodiments of the present invention 10.

As shown in FIGS. 1 and 2, there can be an optional retrieval implement 33 bonded or otherwise attached to one of the wings 12 13 of the present invention 10, such that the sanitary napkin 10 can be removed from a patient's buttocks without manual contact with the absorbent part of the napkin 10. The retrieval implement 33 can be a single string, a loop of string, a molded plastic member, or any similar appendage which will not deform in tension and will facilitate removal of the napkin 10 from between a patient's buttocks.

As shown in FIG. 2, the protective layer 24 can optionally have two flanges 46 and 47 that wrap around the wing edges 41 and 42 in order create a more rigid and robust assembly and prevent the escape of bodily fluids from the absorbent layer 26, thereby avoiding contamination of the patient's skin or clothing.

The preferred method for using the present invention 10 is as follows: Prior to insertion, the invention 10 is first compressed by applying opposing inward forces against the two wings 12 and 13 between one's thumb and forefinger, thereby decreasing the expanded wing width 18 to the compressed wing width 19. The compressed invention 10 is then inserted into the intergluteal cleft between a patient's buttocks such that outer crease 40 engages the perianal area and the opposed wings 12 and 13 engage the buttocks.

The invention is held in place by the force between the buttocks and the opposed wings 12 and 13, which results from the restorative force counteracting the elastic deformation of spring means 11. As a patient's buttocks move and contort to accommodate the activities of daily living, the invention 10 dynamically flexes and adjusts without being dislodged. Thus, the invention 10 remains in place until it is to be removed, at which point a patient either again manually applies a compression force to the wings 12 and 13, or alternately manually applies a pulling force to retrieval implement 33.

What is claimed is:

1. A sanitary napkin comprising:
(a) a semi-rigid, deformable, multi-layered oblong sheet having a medial axis, along which medial axis the sheet has a crease such that it is bent in a “V” or “U” shaped cross-sectional profile to form two opposing wings that extend outward from the medial axis at an acute or obtuse extension angle; and
(b) a spring means, which either comprises one or more discrete structures within the sanitary napkin or is integrated within the overall structure of the napkin, and which spring means exert(s) a restorative force that tends to maintain the extension angle of the wings and to restore the extension angle when the wings are compressed together.

2. The sanitary napkin according to claim 1, wherein the wings have an inner protective layer and an outer absorbent layer, with the protective layer comprising a non-porous, impervious material, and the absorbent layer comprising one or more sub-layers of porous material(s).

3. The sanitary napkin according to claim 2, wherein the absorbent layer comprises two sub-layers: an outer wicking sub-layer comprised of a foam material having relatively small pores, and an inner storage sub-layer having relatively larger pores.

4. The sanitary napkin according to claim 3, wherein one or more sub-layers of the absorbent layer is impregnated with a pharmacologically active agent.

5. The sanitary napkin according to claim 3, wherein both sub-layers of the absorbent layer are impregnated with a pharmacologically active agent, such that the wicking sub-layer more rapidly releases the pharmacologically active agent, while the storage sub-layer more slowly releases the pharmacologically active agent.

6. The sanitary napkin according to any of claims 1-5, wherein each of the spring means has a central pivot point, and the spring means is/are attached to the inner side of the crease, such that the pivot point(s) are aligned with the medial axis.

7. The sanitary napkin according to any of claims 1-5, wherein the spring means comprises a semi-rigid, elastic, deformable layer which is laminated between the protective layer and the absorbent layer, or between the sub-layers of the absorbent layer.

8. The sanitary napkin according to any of claims 1-5, wherein the spring means comprises a restorative elasticity of the oblong sheet itself, such that the oblong sheet is fabricated of a semi-rigid, deformable, elastic material, and such that the elastic material comprising the oblong sheet exerts a restorative force and tends to maintain the extension angle of the wings and to restore the extension angle when the wings are compressed together.
